(19) United States
(12) Patent Application Publication
(10) Pub. No.: US 2022/0249277 A1
(43) Pub. Date: Aug. 11, 2022

(54) FLEXIBLE COLD/HOT COMPRESS STRIP

(71) Applicant: Chin-Hwa HWANG, Taipei City (TW)

(72) Inventor: Chin-Hwa HWANG, Taipei City (TW)

(21) Appl. No.: 17/495,787

(22) Filed: Oct. 6, 2021

(30) Foreign Application Priority Data
Feb. 8, 2021 (TW) 110104666

(51) Int. Cl. A61F 7/02; A61F 7/00

(52) U.S. Cl. CPC A61F 7/02; A61F 7/007; A61F 2007/0226; A61F 2007/0219; A61F 2007/0094; A61F 2007/0247

(57) ABSTRACT

A flexible cold/hot compress strip includes a flexible strip body, and a thermoelectric cooler detachably installed to a side of the strip body. The other side of the strip body has a thermally conductive layer and a flexible contact layer. With the flexible design of the strip body, the thermally conductive layer and the contact layer, the whole cold/hot compress strip can be attached closely to human body for cold or hot compress, and the thermoelectric cooler has a detachable design for easy carry and use.

### FIELD OF THE INVENTION

The present invention relates to a flexible cold/hot compress strip, and more particularly to the cold/hot compress strip that can be flexibly attached to a human body and can fit the body radian of a user for cold or hot compress. The technical contents mainly include the flexibility design, uniformly conducting and distributing heat to entire cold/hot compress strip, and using a thermoelectric cooler system of the cold/hot compress strip with a detachable design for easy carry and use.

### BACKGROUND OF THE INVENTION

Traditional cold and hot compress devices generally use liquid such as cold water or hot water as a heat and cold conduction medium to let the liquid circulate in a preset channel, in order to achieve the purpose of cold compress or hot compress. These devices not just have a relatively large volume only, but also have the drawback that the heat of the cold water or hot water will decrease with the distance of the circulation channel, resulting in a significant temperature difference between the cold or heat generation source and the end of the circulation channel, as well as a long time delay when switching between the cold and hot compress modes, which is inconvenient to use.

As science and technology advance continuously, the volume of thermoelectric cooler chips becomes progressively smaller, and the heating speed becomes increasingly faster, and most of the present existing cold and hot compress devices mainly use the thermoelectric cooler chip as a heat generation source. To overcome the drawbacks of the conventional cold and hot compress devices that use the liquid such as cold water or hot water as a heat and cold conduction medium. For example, R.O.C. Pat. No. M456188 entitled “Cold and hot compress pad” has disclosed a technical measure of using a cross-shaped strip body made of a metal sheet as a cold and hot conduction interface, and the thermoelectric cooler chip is installed at the center of the cross-shaped metal sheet. The cross-shaped metal sheet will tied on a human body during use, so the metal sheet can conduct heat and provide the function of cold compress or hot compress quickly.

However, the structure disclosed in R.O.C. Pat. No. 456188 still inadequate and have room for improvement. Due to the concentrated and rapid transfer of heat to the metal sheet, it is easy to cause the metal sheet to be situated at the assembling position of the thermoelectric cooler chip; and due to the short-distance linear heat transfer, the temperature may be overheated or overcooled. Furthermore, it is also easy for the thermoelectric cooler chip to produce a temperature difference at a position away from the end, and users may feel the non-uniform temperature.

In addition, R.O.C. Pat. No. 456188 uses an elastic bandage to fix the strip body and the metal sheet on a human body. During operation, it is not only easy to cause the problem of the displacement of the elastic bandage, but also hard to effectively stick the strip body to the skin due to the properties of the material of the metal sheet. As a result, users have to make adjustments for the appropriate position repeatedly. Obviously, such application is inconvenient and uncomfortable for users. Further, the aforementioned thermoelectric cooler chip is permanently fixed and cannot be removed from the strip body and installed to another metal sheet for use, which limits the scope of application.

In order to control the cold and hot compress device to a suitable temperature, some commercial products use smart control and limited temperature range to prevent burns or frostbite. However, different users have different tolerances for temperature and using time. Therefore, the need for setting and adjusting the time of use and temperature of the cold and hot compress device still exists.

In view of the aforementioned drawbacks of the prior art, the inventor of the present invention based on years of experience in the related industry to conduct extensive research and experiment, and finally developed a flexible cold/hot compress strip to overcome the problems of the conventional compress strip that uses a metal sheet as a cold and hot conduction interface and fail to attach the compress strip closely with the users' body, and the thermoelectric cooler chip cannot be removed or installed easily, and the conduction of heat is too concentrated, resulting in burns or frostbites.

### SUMMARY OF THE INVENTION

The primary objective of the present invention is to provide a flexible cold/hot compress strip capable of conducting heat uniformly, and a detachable thermoelectric cooler, such that the cold/hot compress strip can match with the radian of a user's body for cold or hot compress, and allow the heat of the thermoelectric cooler on the strip body to be conducted and dissipated uniformly, so as to improve the cold or hot compress effect.

To achieve the aforementioned and other objectives, the present invention discloses a flexible cold/hot compress strip including a flexible strip body and a thermoelectric cooler installed on a side of the strip body, wherein a flexible thermally conductive layer and a flexible contact layer are stacked on the other side of the strip body, and the heat generated by the thermoelectric cooler can be directly or indirectly conducted through the heat conduction layer to the contact layer; the flexible strip body, the thermally conductive layer and the contact layer are attached in correspondence with a human body radian to apply a cold or hot compress to a human body. The heat conduction layer has a heat receiving surface formed at a position corresponding to the thermoelectric cooler for receiving heat, and a heat conduction surface formed at a position other than that of the heat receiving surface. A barrier plate is installed between the heat receiving surface of the heat conduction layer and the contact layer for preventing the heat receiving surface from accumulating the heat generated by the thermoelectric cooler and linearly conducted in a short distance at the relative position of the contact layer, so that the heat can be dispersed outwardly from the center of the heat receiving surface and uniformly conducted to the heat conduction surface and the contact layer, so as to avoid discomfort to human body caused by a part of the contact layer of a too-high or too-low temperature.

The present invention has the following advantages compared with the prior art:

1. In the present invention, the strip body has a barrier plate capable of preventing the heat receiving surface from accumulating the heat generated by the thermoelectric cooler and linearly conducted in a short distance at the relative position of the contact layer at the joint position of the strip body, so that the heat can be dispersed outwardly from the center of the heat receiving surface and uniformly conducted to the heat conduction surface and the contact layer to avoid discomfort to the human body caused by a part of the contact layer of a too-high or too-low temperature.

2. The thermoelectric cooler of the present invention has a design of detachable structure for easy carry and assembly. After the thermoelectric cooler is removed from the strip body, it can be installed to another flexible strip body for use, so as to expand the scope of application.

3. In the present invention, the flexible strip body, the heat conduction layer, and the flexible contact layer are made of a flexible material. While maintaining the effective and uniform conduction of heat, the invention can be fitted to human body radian, thus achieving the effects of convenient use and high comfort.

The technical characteristics of the present invention will become apparent with the detailed description of preferred embodiments accompanied with the illustration of related drawings.

### BRIEF DESCRIPTION OF THE DRAWINGS

FIG. 1 is a perspective view of this invention;

FIG. 2 is an exploded view of a thermoelectric cooler of this invention;

FIG. 3 is an exploded view of a flexible strip body of this invention;

FIG. 4 is a bottom view of a first embodiment of this invention;

FIG. 5 is a cross-sectional view of the first embodiment of this invention;

FIG. 6 is a partial blowup view of the first embodiment of this invention;

FIG. 7 is a cross-sectional view of a second embodiment of this invention;

FIG. 8 is a partial blowup view of the second embodiment of this invention;

FIG. 9 is a bottom view of a third embodiment of this invention;

FIG. 10 is a cross-sectional view of the third embodiment of this invention;

FIG. 11 is a cross-sectional view of a fourth embodiment of this invention

FIG. 12 is a partial blowup view of the fourth embodiment of this invention; and

FIG. 13 is a schematic block diagram of a thermoelectric cooler of this invention.

### DESCRIPTION OF THE PREFERRED EMBODIMENTS

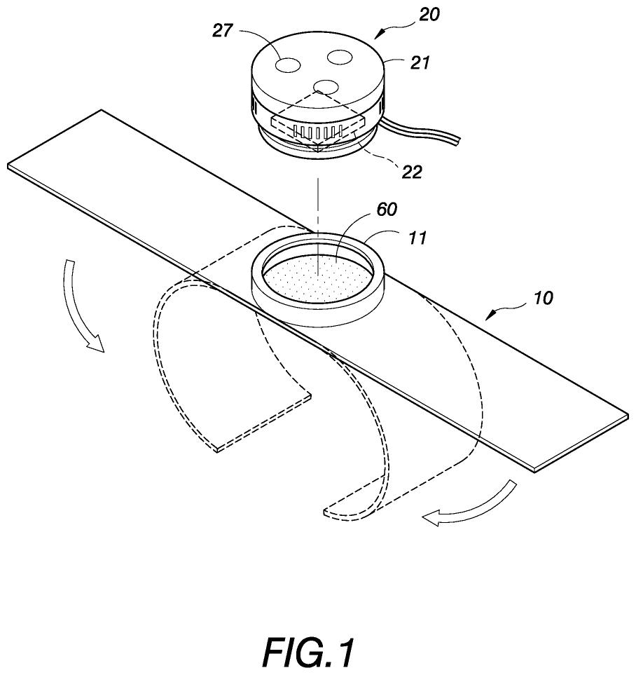

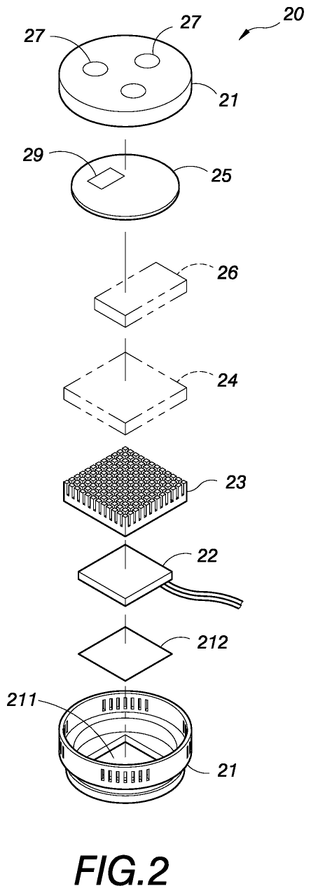

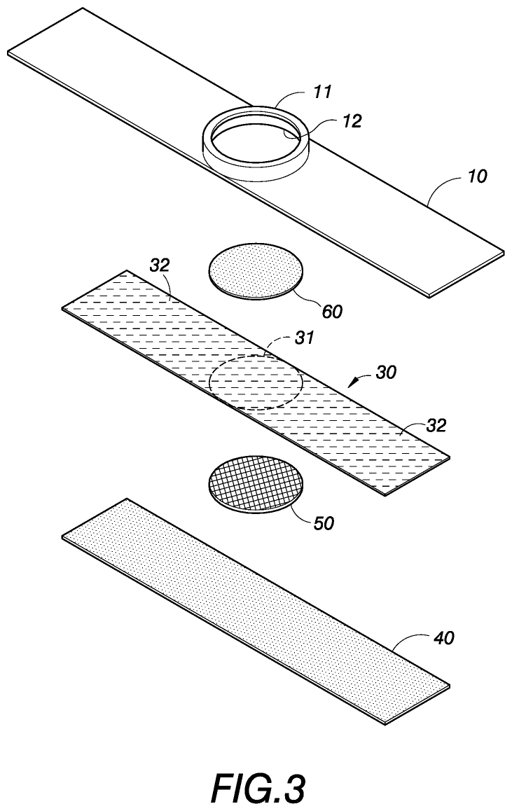

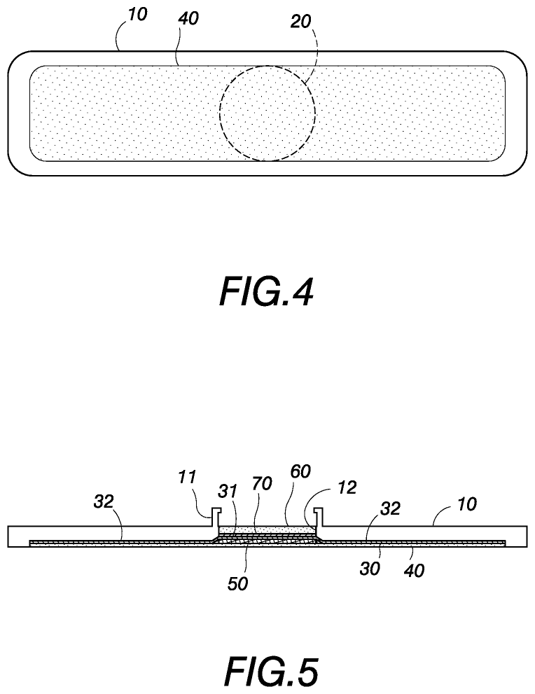

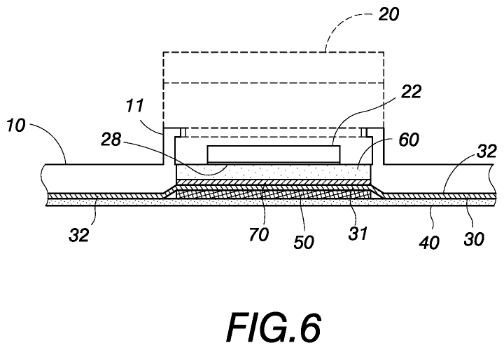

With reference to FIGS. 1 to 6 for a flexible cold/hot compress strip of the present invention, the flexible cold/hot compress strip includes a flexible strip body 10, a thermoelectric cooler system 20 installed on a side of the strip body 10, and a flexible heat conduction layer 30 and a flexible contact layer 40 which are stacked on the other side of the strip body 10.

The thermoelectric cooler system 20 includes a shell 21 with a plurality of vent holes, a thermoelectric cooler chip 22 installed in the shell 21, a cooling fin 23 closely attached to one side of the thermoelectric cooler chip 22, a fan 24 for cooling the cooling fin 23, a control circuit board 25 for controlling the operation of the thermoelectric cooler chip 22, an electric storage body 26 for supplying electric power to the thermoelectric cooler system 20, and an operating interface 27 disposed on a surface of the shell 21 for operating to control the cold and hot temperatures.

The bottom of the shell 21 has a through hole 211, and a thermal conductive plate 212 is mounted on the outer side of the through hole 211, and the other side of the thermoelectric cooler chip 22 is disposed in the through hole 211 and closely attached to the thermal conductive plate 212 to form a heat generating and cooling part 28 on a surface of the shell 21.

Users can operate on the operating interface 27 of the thermoelectric cooler system 20 to select the option of automatically switching to the cold compress, hot compress, or cold/hot compress, and the control circuit board 25 is provided for controlling the operation of the thermoelectric cooler chip 22, while driving the fan 24 to dissipate the heat generated by the heat generating and cooling part 28.

The heat generated by the heat generating and cooling part 28 of the thermoelectric cooler system 20 can be directly or indirectly conducted to the contact layer 40 through the heat conduction layer 30, and the flexible strip body 10, heat conduction layer 30 and contact layer 40 are matched and attached closely with a human body radian to apply the cold or hot compress to a human body.

The heat conduction layer 30 includes a heat receiving surface 31 configured to be corresponsive to the assembling position of the thermoelectric cooler system 20 and provided for receiving heat, a heat conduction surface 32 disposed at a position other than that of the heat receiving surface 31, and a barrier plate 50 installed at a position between the heat receiving surface 31 of the heat conduction layer 30 and the contact layer 40, wherein the barrier plate 50 has an area substantially equal to the area of the heat receiving surface 31.

The barrier plate 50 is provided for preventing the heat receiving surface 31 from accumulating the heat generated by the thermoelectric cooler 20 and linearly conducted in a short distance at the relative position of the contact layer 40, so that the heat can be dispersed outwardly from the center of the heat receiving surface 31 and uniformly conducted to the heat conduction surface 32 and the contact layer 40, so as to avoid discomfort to human body caused by a part of the contact layer 40 of a too-high or too-low temperature, and improve the drawback of the prior art that the thermoelectric cooler chip conducts the heat of the metal sheets in a too-concentrated manner

In the figures, one side of the strip body 10 has a protruding positioning ring 11, and an opening 12 formed at the inner periphery of the positioning ring 11 and configured to be corresponsive to the heat receiving surface 31 of the heat conduction layer 30.

The positioning ring 11 is provided for detachably embedding the thermoelectric cooler system 20 into the inner periphery for positioning, and the heat generating and cooling part 28 is configured to be corresponsive to the inner side of the opening 12, so that the heat generated by the heat generating and cooling part 28 can be directly or indirectly conducted to the heat receiving surface 31 of the heat conduction layer 30.

In an embodiment, the inner periphery of the positioning ring 11 has a hook for keeping the shell 21 of the thermoelectric cooler system 20 at the inner periphery of the positioning ring 11 to help fixing the thermoelectric cooler system 20 onto strip body 10.

In an embodiment, the strip body 10 is made of a silicone material capable of preventing heat dissipation, but the shell 21 of the thermoelectric cooler system 20 is made of a harder material, so that the strip body 10 has a thermally conductive gel patch 60 installed on the inner side of the opening 12 and configured to be opposite to the heat receiving surface 31 of the heat conduction layer 30 to prevent the thermoelectric cooler system 20 from being loosened or falling out during use, wherein the thermally conductive gel patch 60 not just can conduct the heat of the heat generating and cooling part 28 only, but also can allow the heat generating and cooling part 28 of the thermoelectric cooler system 20 to be attached and fixed due to the self-adhesiveness of the thermally conductive gel patch 60.

With the aforementioned structure, the thermoelectric cooler system 20 is designed with a detachable structure, and the positioning ring 11 and the thermally conductive gel patch 60 are assembled and fixed onto the flexible strip body 10 to prevent the strip body 10 from being loosened or falling out from the thermoelectric cooler system 20 during use. After the thermoelectric cooler system 20 is removed from the strip body 10, it can be installed to another flexible strip body 10 of a different model or a different size, so as to expand the scope of application. In addition, the thermoelectric cooler system 20 has an electric storage body 26 installed therein to allow the thermoelectric cooler system 20 to be operated without utility power, which can also improve the convenience of carry and use.

In an embodiment, the strip body 10 has a thermally conductive metal sheet 70 installed between the thermally conductive gel patch 60 and the heat receiving surface 31 of the heat conduction layer 30, wherein the thermally conductive metal sheet 70 is a copper sheet or an aluminum sheet that can help conducting heat and maintaining a smooth surface of the strip body 10.

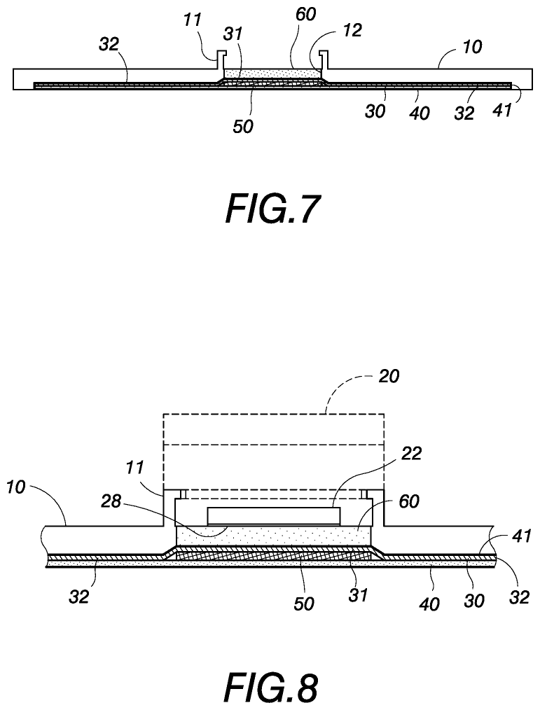

In another implementation as shown in FIGS. 7 and 8, a cladding layer 41 is formed on the exterior of the heat conduction layer 30, the barrier plate 50, and the contact layer 40; the cladding layer 41 has a little effect on the thermal resistance of the contact layer 40. If the thermally conductive metal sheet (not shown in the figures) is omitted, there is still a good cold/hot compress effect after the cladding layer 41 is set. Alternatively, the cladding layer 41 can be formed on the exterior of the heat conduction layer 30 and the barrier plate 50, and the cladding layer 41 not just can protect the heat conduction layer 30 only, but can further protect the heat conduction layer 30 from being peeled, scratched, broken or damaged when the heat conduction layer 30 is made of a flexible graphite sheet.

In an embodiment, the heat conduction layer 30 is made of flexible graphite sheet, hydrogel sheet, graphene gel sheet, or copper clad graphite sheet, etc., and the contact layer 40 is made of a flexible graphite sheet, a self-adhesive hydrogel patch, or a self-adhesive hydrogel patch for medical treatment, etc.

With the aforementioned variety of materials, there are different embodiments of the flexible cold/hot compress strip. For example, the heat conduction layer 30 can be a graphite sheet and the contact layer 40 can be a hydrogel patch, or the heat conduction layer 30 can be a hydrogel sheet and the contact layer 40 can be a hydrogel patch. The hydrogel patch is skin-friendly and self-adhesive. In addition, the strip body, graphite sheet, and hydrogel patch are all made of flexible materials, so that they can directly fit the human body curve of the user's body and conduct heat. Obviously, the invention is convenient and comfortable to use. In addition, after the flexible cold/hot compress strip has been used for a long time, the hydrogel patch can be peeled off and replaced, so as to provide a convenient and sanitary use.

In actual application, users can place medicine at a position between a part of their body and the hydrogel patch of the contact layer 40 to achieve the effect of medical treatment, and the heat can be controlled to apply or stop the medication in order to achieve better treatment and healthcare effects, or the hydrogel patch of the contact layer 40 is directly used for medical treatment, wherein its polymer structure under the heat control functionally implement the drug release to achieve the good treatment and healthcare effects.

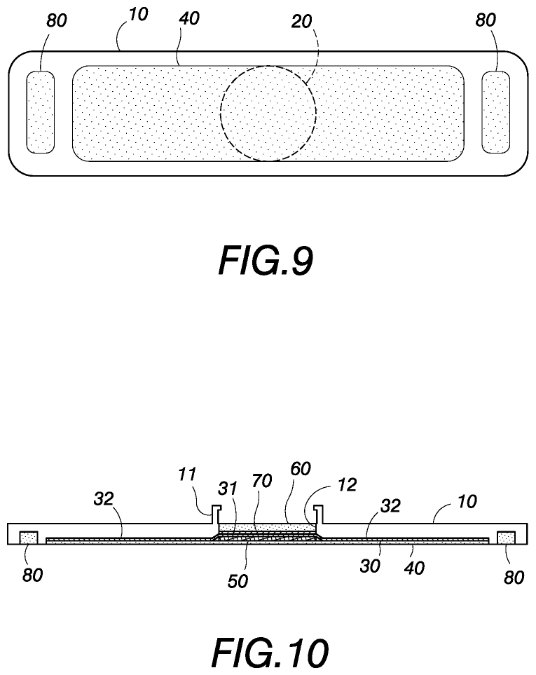

In FIGS. 9 and 10, the aforementioned materials are applied in this embodiment of this invention. For example, both of the heat conduction layer 30 and contact layer 40 are made of graphite sheets, and the contact layer 40 has a width smaller than the strip body 10, and both sides of the strip body 10 relative to the contact layer 40 have a hydrogel patch 80 that can be attached to a human body, and the adhesiveness of the hydrogel patch 80 allows the strip body 10 to be attached and fixed onto human skin.

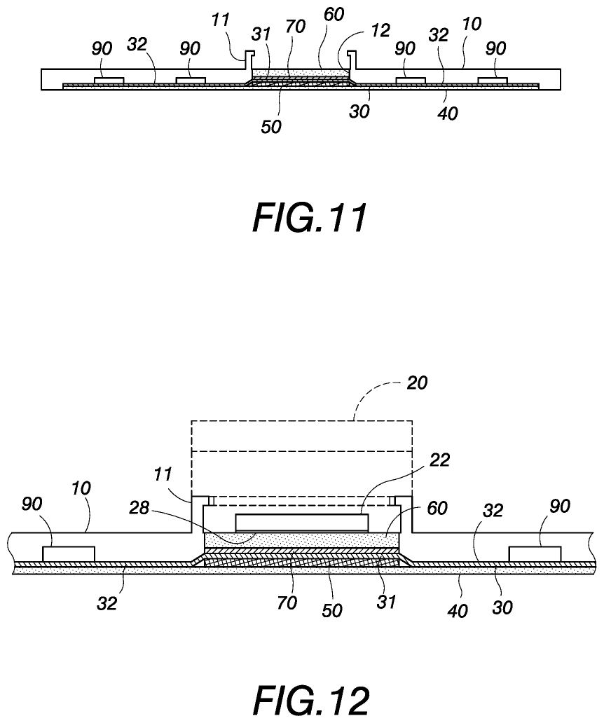

In FIGS. 11 and 12, a plurality of energy-saving metal sheets 90 are installed between the strip body 10 and the heat conduction surface 32 of the heat conduction layer 30, wherein the energy-saving metal sheets 90 are copper sheets or aluminum sheets. The energy-saving metal sheets 90 can store the heat coming from the heat conduction surface 32 and adjusting the temperature of the heat conducted to the contact layer 40, so as to avoid the possible quick heat loss caused by the high conductivity of the graphite sheets.

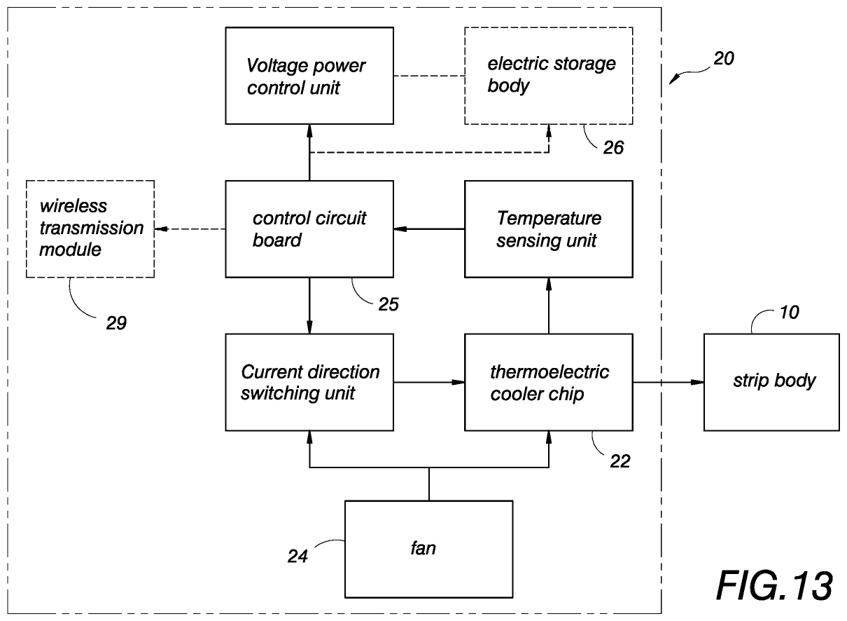

In FIG. 13, the control circuit board 25 further includes a wireless transmission module 29 for transmitting signals with an external mobile device, wherein the wireless transmission module 29 is a Bluetooth or Wi-Fi device that allows the thermoelectric cooler system 20 to connect to the external mobile device for signal transmission through the wireless transmission module 29. The external mobile device can have a compatible APP installed to the thermoelectric cooler system 20, and the APP can be run to provide the functions of controlling the temperature, setting the time of use, and managing the health information and cloud health inspection.

While the invention has been described by means of specific embodiments, numerous modifications and variations could be made thereto by those skilled in the art without departing from the scope and spirit of the invention as set forth in the claims.

What is claimed is:

1. A flexible cold/hot compress strip, comprising a flexible strip body, and a thermoelectric cooler installed on a side of the strip body, characterized in that:
a flexible thermally conductive layer and a flexible contact layer are stacked on the other side of the strip body, and the heat generated by the thermoelectric cooler can be directly or indirectly conducted through the heat conduction layer to the contact layer, and the flexible strip body, the thermally conductive layer and the contact layer are attached in correspondence with a human body curve to apply a cold or hot compress to a human body; and
the heat conduction layer comprises a heat receiving surface formed at a position corresponding to the thermoelectric cooler for receiving heat, a heat conduction surface formed at a position other than that of the heat receiving surface, a barrier plate installed between the heat receiving surface of the heat conduction layer and the contact layer for preventing the heat receiving surface from accumulating the heat generated by the thermoelectric cooler and linearly conducted in a short distance at the relative position of the contact layer, so that the heat can be dispersed outwardly from the center of the heat receiving surface and uniformly conducted to the heat conduction surface and the contact layer, so as to avoid discomfort to the human body caused by a part of the contact layer of a too-high or too-low temperature.

2. The flexible cold/hot compress strip according to claim 1, wherein the thermoelectric cooler has a heat generating and cooling part, and one side of the strip body has a positioning ring protruding therefrom, and the inner periphery of the positioning ring has an opening formed thereon and configured to be corresponsive to the heat receiving surface of the heat conduction layer;
the positioning ring is provided for detachably embedding the thermoelectric cooler detachably into the inner periphery for positioning, and the heat generating and cooling part is configured to be corresponsive to the inner side of the opening, so that the heat generated by the heat generating and cooling part can be directly or indirectly conducted to the heat receiving surface of the heat conduction layer.

3. The flexible cold/hot compress strip according to claim 2, wherein the strip body has a thermally conductive gel patch disposed on the inner side of the opening and configured to be corresponsive to the heat receiving surface of the heat conduction layer, and the heat generating and cooling part of a thermoelectric cooler system 20 is attached closely to the thermally conductive gel patch and fixed into a position.

4. The flexible cold/hot compress strip according to claim 3, wherein the strip body has a thermally conductive metal sheet installed between the thermally conductive gel patch and the heat receiving surface of the heat conduction layer.

5. The flexible cold/hot compress strip according to claim 1, wherein the heat conduction layer, the barrier plate, and the contact layer are covered by a cladding layer, or the heat conduction layer and the barrier plate are covered by a cladding layer.

6. The flexible cold/hot compress strip according to claim 1, wherein the contact layer has a width smaller than the strip body, and the strip body has a hydrogel patch disposed on both lateral sides of the heat conduction layer and attached closely to the human body.

7. The flexible cold/hot compress strip according to claim 1, further comprising a plurality of energy-saving metal sheets spaced apart from one another and installed between the strip body and the heat conduction surface of the heat conduction layer for storing heat.

8. The flexible cold/hot compress strip according to claim 1, wherein the strip body is made of a silicone material; the heat conduction layer is made of a flexible graphite sheet or a hydrogel sheet with a high coefficient of thermal conductivity; the contact layer is made of a flexible graphite sheet, a self-adhesive hydrogel patch, or a self-adhesive hydrogel patch used for medical treatment.

9. The flexible cold/hot compress strip according to claim 1, wherein the heat receiving surface has an area substantially equal to the area of the barrier plate.

10. The flexible cold/hot compress strip according to claim 2, wherein the thermoelectric cooler comprises a shell with a plurality of vent holes, and the shell comprises a thermoelectric cooler chip installed therein, a cooling fin attached on one side of the thermoelectric cooler chip, a fan for cooling the cooling fin, a control circuit board for controlling the operation of the thermoelectric cooler chip, and an electric storage body for supplying the electric power required for the operation of the thermoelectric cooler system 20, and the control circuit board comprises a wireless transmission module for transmitting signals with an external mobile device, and an operating interface installed on a surface of the shell for setting the operating cold/hot temperature, and the bottom of the shell has a through hole, and the outer side of the through hole has a thermal conductive plate, and the other side of the thermoelectric cooler chip is disposed in the through hole and attached closely to the thermal conductive plate to form the heat generating and cooling part on a surface of the shell.
